(19) United States
(12) Patent Application Publication
(10) Pub. No.: US 2025/0222203 A1
(43) Pub. Date: Jul. 10, 2025

(54) AUTOMATIC INJECTION DEVICE

(71) Applicant: ASAHI KASEI PHARMA CORPORATION, Tokyo (JP)

(72) Inventor: Mayumi KUMAGAI, Tokyo (JP)

(73) Assignee: ASAHI KASEI PHARMA CORPORATION, Tokyo (JP)

(21) Appl. No.: 19/088,211

(22) Filed: Mar. 24, 2025

Related U.S. Application Data
(62) Division of application No. 17/639,376, filed on Mar. 1, 2022.

(30) Foreign Application Priority Data
Sep. 3, 2019 (JP) 2019-160374

(51) Int. Cl. A61M 5/20; A61M 5/31

(52) U.S. Cl. CPC A61M 5/20; A61M 5/31

(57) ABSTRACT

A two-step type automatic injection device including a means of automatically subcutaneously administering a drug solution with pressing a pressing member provided at one of end portions against an administration site, the automatic injection device including a means of preventing or controlling from placement of a finger on an end portion thereof. The automatic injection device of the present invention is very useful in pharmaceutical industries.

### CROSS-REFERENCE TO RELATED APPLICATIONS

This application is a Divisional application of co-pending application Ser. No. 17/639,376, filed on Mar. 1, 2022, which is the National Phase under 35 U.S.C. § 371 of International Application No. PCT/JP2020/029436, filed on Jul. 31, 2020, which claims the benefit under 35 U.S.C. § 119 (a) to Patent Application No. 2019-160374, filed in Japan on Sep. 3, 2019, all of which are hereby expressly incorporated by reference into the present application.

### TECHNICAL FIELD

The present invention relates to an automatic injection device and a method of preventing or controlling mis-operations thereof, and a label or the like adhered to the automatic injection device.

### BACKGROUND ART

As methods of administering a pharmaceutical, methods of injection administration such as intravenous, subcutaneous, or intramuscular injections have been known. Commonly, physicians or nurses perform injections, but patients themselves may perform injections at home with an automatic injection device (which may be also called auto-injector) to administer a drug, for a certain kind of pharmaceuticals, for the purpose of the treatment of diseases. At that time, some patients may be averse to self-injections if the patients have a fear of needles. The fear of needles may be understood as one of the symptoms of phobias such as needle phobia, belonephobia, and aichmophobia.

In the recent years, two types of the automatic injection devices have been proposed. One is a three-step type automatic injection device which typically comprises a needle, a needle cover for protecting a needle, a cap attached to the needle and the needle cover, and an injection button of a drug, in which the needle cover is locked at the time point of removing the cap, and the injection button is not activated unless the device is pressed against skin. Therefore, the automatic injection device necessitates three operations of 1 “removing a cap,” 2 “pressing against skin (injection site),” and 3 “pushing an injection button.” The three-step type automatic injection device as described above has been disclosed in Patent Publications 1, 3, and the like, and some automatic injection devices are practically used (Non-Patent Publications 1 to 3).

The other is a two-step type automatic injection device, which typically comprises a needle, a needle cover for protecting the needle, and a cap attached to the needle and the needle cover, in which the needle cover also serves as an injection button of a drug. Therefore, the drug is automatically injected with two operations of 1 “removing a cap” and 2 “pressing against skin (injection site).” The two-step type automatic injection device as described above has been disclosed in Patent Publications 1, 3, and the like, and some automatic injection devices are practically used (Non-Patent Publications 4 and 5).

### PRIOR ART REFERENCES

### Patent Publications

Patent Publication 1: Japanese Unexamined Patent Publication No. 2018-531702
Patent Publication 2: Japanese Patent Laid-Open No. 2019-88873
Patent Publication 3: Japanese Patent Gazette No. 5758860

### Non-Patent Publications

Non-Patent Publication 1: PDA Journal of GMP and Validation in Japan 19 (2), 30-38 (2017)
Non-Patent Publication 2: ENBREL Subcutaneous Injection 50 mg PEN 1.0 mL, Pharmaceutical Guides for Patients
Non-Patent Publication 3: ORENCIA Subcutaneous Injection 125 mg Autoinjector, Pharmaceutical Guides for Patients
Non-Patent Publication 4: Folia Pharmacol. Jpn. 151, 261-272 (2018)
Non-Patent Publication 5: Etanercept BS Subcutaneous Injection 50 mg PEN 1.0 mL, “TY,” Pharmaceutical Guides for Patients

### SUMMARY OF THE INVENTION

### Problems to be Solved by the Invention

The automatic injection device of the type in which a drug solution is automatically administered by the operations of 1. removing a cap (a cap), and 2. pressing a tip portion 136 as a pressing member against an administration site (hereinafter also called “a two-step type” or “a two-step type automatic injection device), as described in Patent Publication 2, is simpler in the operations than the automatic injection device further requiring 3. pushing a button (hereinafter also called “a three-step type” or “a three-step type automatic injection device”), as disclosed in Patent Publications 1 and 3. Therefore, the two-step type is highly convenient for the users.

An object of the present invention is to provide an automatic injection device, a method of administration or the like, capable of maintaining the conveniences for the users of having a smaller number of administration steps in the two-step type automatic injection device than that of the three-step automatic injection device, and further being even more safely administrable.

More specifically, an object of the present invention is to provide a two-step type automatic injection device for preventing or controlling mis-operations of undesirably administering to a finger, a method for preventing or controlling mis-operations of undesirably administering to a finger in the two-step type automatic injection device, and the like.

### Means to Solve the Problems

The present inventors have noticed that in a two-step type automatic injection device, there is a risk of undesirably administering a drug mistakenly by undesirably pressing a pressing member of the same device against a site other than an appropriate administration site, and intensively studied the causations thereof. As a result, surprisingly, the present inventors have newly found out that there are some possible risks of mis-operations of erroneously administering to a finger by misunderstanding the pressing member pressed against the administration site as a button in the three-step type, or the like.

In view of the above, as a result of intensive studies of the mechanism for providing a safer administration by reducing the possible risks of mis-operations, the present inventors have found out that the undesirably risks of mis-operations can be reduced because a two-step type automatic injection device includes a means of preventing or controlling from placement of a finger on an end portion thereof.

Further, the present inventors have found out that the possible risks of mis-operations can be reduced because a two-step type automatic injection device includes a means of guiding for gripping the automatic injection device in a direction of placing a thumb towards an end portion thereof, not including a pressing member.

The present invention relates to the following [1] to [9]:
[1] A two-step type automatic injection device including a means of automatically subcutaneously administering a drug solution with pressing a pressing member provided at one of end portions against an administration site, the automatic injection device including a means of preventing or controlling from placement of a finger on an end portion thereof.
[2] The automatic injection device according to [1], wherein an indication of gripping not to place a finger on an end portion of the automatic injection device is attached as the means of preventing or controlling from placement of a finger on an end portion of the automatic injection device.
[3] A two-step type automatic injection device including a means of automatically subcutaneously administering a drug solution with pressing a pressing member provided at one of end portions against an administration site, the automatic injection device including a means of guiding for gripping the automatic injection device in a direction of placing a thumb towards an end portion thereof not including the pressing member.
[4] The automatic injection device according to [3], wherein an indication of either of the end portions including the pressing member is attached as the means of guiding for gripping the automatic injection device in a direction of placing a thumb towards an end portion thereof not including the pressing member.
[5] The automatic injection device according to any one of [1] to [4], which is applicable to a female patient.
[6] A method for preventing or controlling mis-operations of a two-step type automatic injection device including a means of automatically subcutaneously administering a drug solution with pressing a pressing member provided at one of end portions against an administration site, the method including applying a means of preventing or controlling from placement of a finger on an end portion of the automatic injection device to prevent or control mis-operations thereof.
[7] A method for preventing or controlling mis-operations of a two-step type automatic injection device including a means of automatically subcutaneously administering a drug solution with pressing a pressing member provided at one of end portions against an administration site, the method including applying a means of guiding for gripping the device in a direction of placing a thumb towards an end portion of the automatic injection device not including the pressing member to prevent or control mis-operations thereof.
[8] A label adhered to a two-step type automatic injection device including a means of automatically subcutaneously administering a drug solution with pressing a pressing member provided at one of end portions against an administration site, wherein an indication of gripping not to place a finger on an end portion of the automatic injection device is attached.
[9] A label adhered to a two-step type automatic injection device including a means of automatically subcutaneously administering a drug solution with pressing a pressing member provided at one of end portions against an administration site, wherein an indication of either of the end portions including the pressing member is attached.

### Advantageous Effects of the Invention

According to the present invention, a two-step type automatic injection device for preventing or controlling mis-operations of undesired administration to a finger, and a method for preventing or controlling mis-operations of undesired administration to a finger in the two-step type automatic injection device can be provided.

### BRIEF DESCRIPTION OF THE DRAWINGS

FIG. 1 The figure shows a schematic of a partial cross-section in an unused state as one embodiment of the automatic injection device of the present invention.

FIG. 2 The figure shows one embodiment of the automatic injection device of the present invention.

FIG. 3 The figure shows one embodiment of the automatic injection device of the present invention.

FIG. 4 The figure shows a label used in Examples.

FIG. 5 The figure shows a label used in Examples.

### MODES FOR CARRYING OUT THE INVENTION

As a result of intensive studies on the above problems, the present inventors have remarked that to begin with mis-operations of undesired administration to a finger would not be caused unless the finger is placed on an end portion of the automatic injection device even if a direction of pressing against an administration site is mistaken. Therefore, the automatic injection device of the present invention features in that the automatic injection device includes a means of preventing or controlling from placement of a finger on an end portion thereof (hereinafter also referred to as “a finger placement preventing or controlling means”). In addition, the present invention is to provide a method for preventing or controlling mis-operations of an automatic injection device, including applying a finger placement preventing or controlling means to prevent or control the mis-operations.

Further, as a result of intensive studies from a different viewpoint on the above problems, the present inventors have remarked that to begin with mis-operations of undesired administration to a finger would not be caused unless a direction of pressing against an administration site is mistaken by gripping the automatic injection device in a correct direction, even if a finger is placed on an end portion. Therefore, the automatic injection device of the present invention features in that the automatic injection device includes a means of guiding for gripping the device in a direction of placing a thumb towards an end portion thereof not including the pressing member (hereinafter also referred to as “a pressing direction guiding means”). In addition, the present invention is to provide a method for preventing or controlling mis-operations of an automatic injection device, including applying a pressing direction guiding means to prevent or control the mis-operations.

The automatic injection device of the present invention can take a structure similar to known two-step type automatic injection devices, except that the automatic injection device includes a finger placement preventing or controlling means or a pressing direction guiding means.

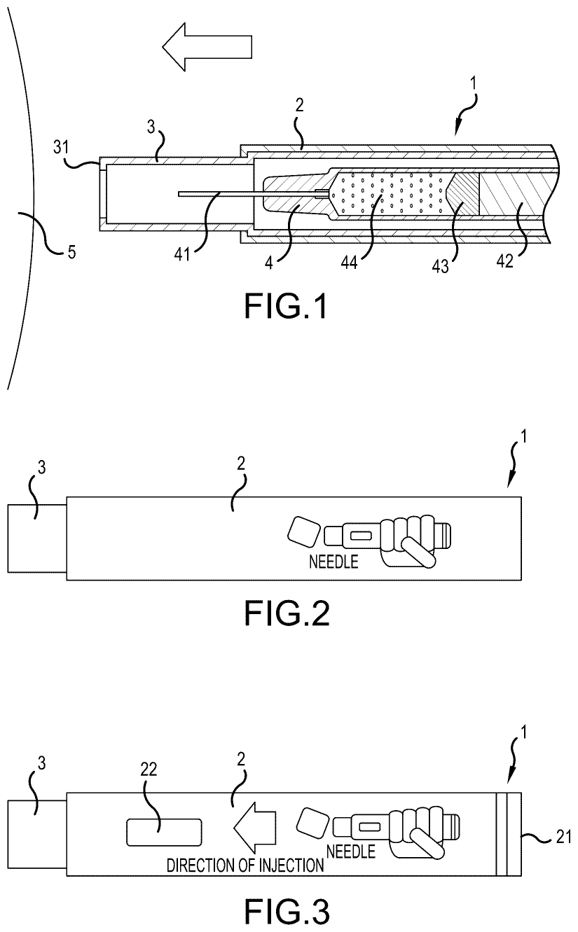

Specifically, the details of outer shapes or internal structures do not matter so long as the automatic injection device includes a means of automatically subcutaneously administering a drug solution with pressing a pressing member provided at one of end portions against an administration site, and for example, embodiments as disclosed in Patent Publication 2 are exemplified. As shown in FIG. 1, there is an embodiment in which in an unused state a pre-filled syringe 4 is housed in a cylindrical sheath 2 in a state that an injection needle 41 is projected in a longitudinal direction, and a needle cover 3 extending along the longitudinal direction from an end portion of the sheath 2 covers the projected injection needle 41 in a radial direction. Here, in FIG. 1, the details of the supporting structure of the pre-filled syringe 4, the members for holding the needle cover 3 at a position of FIG. 1 in an unused state or after use, and the like are not illustrated in the figure. In this embodiment, a tip portion 31 of the needle cover 3 serving as a pressing member is pressed to skin 5 of a patient which is to be an administration site in a direction of the arrow, so that the needle cover 3 is pressed into the sheath 2, and at the same time the injection needle 41 relatively moves forward in the direction of the arrow to pierce into the skin 5 of a patient which is to be an administration site. In addition, although not illustrated in the figure, the moving regulations of a gasket pushing member 42 are released at the same time of pressing, and the gasket pushing member 42 is pressed with an accessory member such as a coil spring to press a gasket 43 in the pre-filled syringe 4, thereby injecting a drug solution 44 in the pre-filled syringe 4 into a live body through the injection needle 41. As described above, in this embodiment, the administration can be made simply by removing a cap covering a needle cover 3 (not illustrated in the figure) and then pressing a tip portion 31 to skin 5 of a patient which is to be an administration site in a direction of the arrow.

The finger placement preventing or controlling means includes, for example, an embodiment in which an indication of gripping not to place a finger on an end portion of the automatic injection device on an outer surface of the sheath 2 is attached as shown in FIG. 2, an embodiment in which an indication showing that a way of gripping of placement of a finger on an end portion of the automatic injection device is not correct is attached, such as an x mark indicated for a way of gripping in which a finger is placed on an end portion of the automatic injection device though not being illustrated in the figure, or the like. Here, as the embodiments of attaching an indication on an outer surface of a sheath 2, these indications may be directly printed or the like to an outer surface of a sheath 2, or a label with these indications may adhered thereto. Therefore, the present invention also provides a label adhered to the automatic injection device.

Further, the finger placement preventing or controlling means includes, for example, besides the indications as mentioned above, an embodiment in which the outer shape of the automatic injection device is made into a shape so that a finger is easily placed according to the recommended way of gripping. The embodiment of attaching indications as mentioned above is preferred, from the viewpoint of costs.

The automatic injection device of the present invention may also include, in addition to, or in an alternative of, the finger placement preventing or controlling means, a pressing direction guiding means. The pressing direction guiding means is preferably, for example, as in the arrow design indicated at the central portion of FIG. 3 (or a combination with printing of the direction of injection), an embodiment of an indication of the direction of pressing against an administration site. Alternatively, it may be an embodiment of an indication of guiding that a tip portion of a needle cover, a needle cover, or a needle covered by the needle cover which serves as a pressing member is present in either of the end portions. Here, the indication of the way of gripping shown in FIG. 3 is a finger placement preventing or controlling means, and a direction of pressing is shown therein, which also is an indication of guiding a direction of placement of a thumb on the side of an end portion 21 not including the pressing member and gripping the automatic injection device, so that the indication is also a pressing direction guiding means. Therefore, the automatic injection device of FIG. 3 includes both means of the finger placement preventing or controlling means and the pressing direction guiding means.

As to the finger placement preventing or controlling means and the pressing direction guiding means, each embodiment may be combined in any manner. As the outer shape of the automatic injection device, any shapes such as nearly quadrangular prism shapes and nearly cylindrical shapes, or the like can be adopted other than a case where the outer shape of the automatic injection device is formed in the shape in which a finger is easily placed according to a recommended way of gripping. For example, in the embodiment of a nearly quadrangular prism shape, when an indication concerning a finger placement preventing or controlling means or an indication concerning a pressing direction guiding means is attached, the indications may be given on one side or plural sides. In addition, when an automatic injection device is used, it may be confirmed from a confirmation window that the administration can be normally performed, and an embodiment in which these indications are given on the side including the confirmation window 22 as in FIG. 3 is preferred.

The patients that are applied with the automatic injection device of the present invention are not particularly limited, and the automatic injection device is particularly suitable for patients with a weak grip, patients with handicaps on hands, female patients, aged patients, and the like. The therapeutic applications of a drug solution provided by the automatic injection device of the present invention are not particularly limited, which include inflammatory diseases such as rheumatoid arthritis and juvenile idiopathic arthritis; metabolic and endocrine diseases such as osteoporosis and diabetes, and diseases distinctively for women such as cervical cancer, ovarian tumors, and breast cancer. As to osteoporosis, the prevalence increases with aging, and women are more likely to be prevalent than men, so that osteoporotic patients can be preferably exemplified as the patients that are applied with the automatic injection device of the present invention.

The automatic injection device of the present invention can be previously filled with a drug solution. The drug solution that is filled is not particularly limited, and preferred examples include tocilizumab, etanercept, abatacept, denosmab, romosozumab, teriparatide, teriparatide acetate and the like.

The automatic injection device containing teriparatide or an acetate thereof in a drug solution as described above is suitably used as an osteoporosis therapeutic agent. In general, the patients subjected to the osteoporosis treatment are aged patients and female patients in many cases, so that the possible risks of mis-operations mentioned above would be considered even higher. However, it can be expected that the mis-operations can be prevented or controlled by using the automatic injection device of the present invention.

### Examples

The present invention will be explained more specifically by means of Examples. However, the present invention is not intended to be bound to the following Examples, and the present invention can be carried out in any embodiments within the scope that does not depart from the spirit of the present invention n.

### 1. Test Method

A confirmation test for administration operations of automatic injection devices was carried out with the following two kinds A, B of automatic injection devices, which were two-step type automatic injection devices each including a means of automatically subcutaneously administering a drug solution with pressing a pressing member provided at one of end portions against an administration site. Here, for the test, those not filled with a drug solution were used in the automatic injection devices A, B.

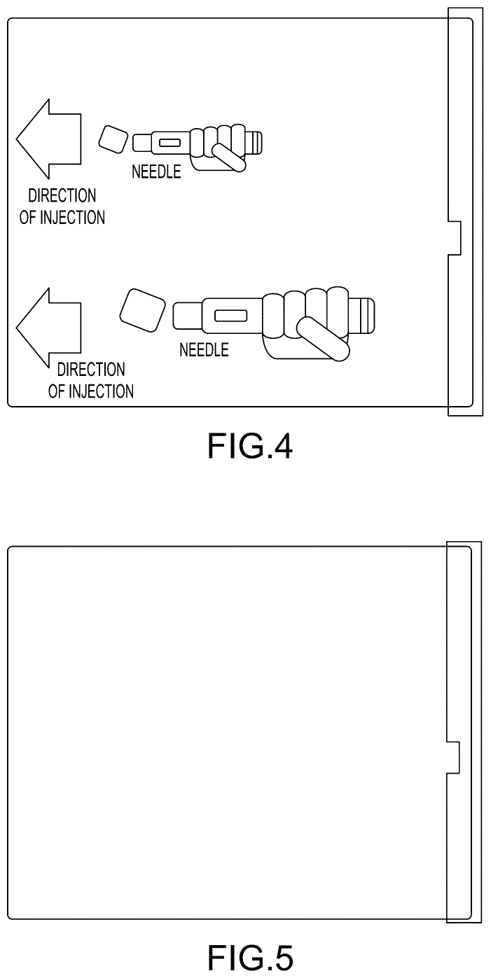

An automatic injection device A: A nearly cylindrical automatic injection device to which a label of FIG. 4 is adhered as 1) a finger placement preventing or controlling means and 2) a pressing direction guiding means (an automatic injection device shown in FIG. 3).

An automatic injection device B: A nearly cylindrical automatic injection device in which a label of FIG. 5 without indications is adhered.

Test subjects for the confirmation test for administration operations were of ages 20 years or older and 70 years or younger, a total of 80 men and women who expressed their consents to the participation to the test by their free will. However, the following individuals were excluded from the test subjects: Those individuals who have previously visually or audially given the explanation concerning the operations of the automatic injection devices A, B, those individuals with some visual disorders that would be difficult to discern the label, those individuals with some disorders that affect the manual operations with fingers that would undesirably pose limitations for gripping, and those individuals who could not understand the contents of the confirmation tests.

Eighty test subjects for the confirmation test for administration operations were randomly assigned into an illustration group (a group of test subjects operating the automatic injection device A) and a control group (a group of test subjects operating the automatic injection device B). In the confirmation test for administration operations, a rating person participated. The rating person explained the intent of the confirmation test for administration operations to each test subject and asked each test subject to read a simple instruction manual and grip the automatic injection device to be administered without previously instructing the operations and the way of gripping the automatic injection device. The rating person recorded whether each test subject placed a finger on an end portion of the automatic injection device.

### 2. Test Results

In a case where a finger is placed on an end portion of the automatic injection device, it was recorded as mis-operations of “placing finger.” The results of recording are as shown in Tables 1 to 3.

TABLE 1
Recording results of the entire 80 test subjects as subjects

| | | Number of |
|---|---|---|
| | | test subjects with |
| | | mis-operations, % |
| | | Placing finger |
| Illustration | Present, illustration group | 4 persons, 10% |
| | Absent, control group | 9 persons, 24% |

TABLE 2
Recording results of only men as subjects
out of the 80 test subjects

| | | Number of |
|---|---|---|
| | | test subjects with |
| | | mis-operations, % |
| | | Placing finger |
| Illustration | Present, illustration group | 4 persons, 13% |
| | Absent, control group | 6 persons, 29% |

TABLE 3
Recording results of only women as subjects
out of the 80 test subjects

| | | Number of |
|---|---|---|
| | | test subjects with |
| | | mis-operations, % |
| | | Placing finger |
| Illustration | Present, illustration group | 0 persons, 0% |
| | Absent, control group | 3 persons, 18% |

As shown in Tables 1 to 3, the illustration group including a finger placement preventing or controlling means according to the present invention had a remarkably low number of test subjects, %, conducting mis-operations of placing a finger, as compared to the control group without including the finger placement or controlling means. In addition, although no test subjects that gripped in a wrong direction were found in both the illustration group and the control group, since the illustration group included a pressing direction guiding means, it was much voiced that the correct direction could be easily recognized. On the other hand, the test subjects would take a longer time to figure out the correct direction in the control group, so that it was much voiced that the indications of the illustration are needed.

### INDUSTRIAL APPLICABILITY

The automatic injection device of the present invention is very useful in pharmaceutical industries.

### EXPLANATION OF NUMERICAL SYMBOLS

1 an automatic injection device
2 a sheath
21 an end portion not including a pressing member
22 a confirmation window
3 a needle cover
31 a tip portion
4 a pre-filled syringe
41 an injection needle
42 a gasket pushing member
43 a gasket
44 a drug solution
5 skin of a patient to be an administration site

1. A label adhered to a two-step type automatic injection device comprising a means of automatically subcutaneously administering a drug solution with pressing a pressing member provided at one of end portions against an administration site, wherein an indication of gripping not to place a finger on an end portion of the automatic injection device is attached.

2. A label adhered to a two-step type automatic injection device comprising a means of subcutaneously automatically administering a drug solution with pressing a pressing member provided at one of end portions against an administration site, wherein an indication of either of the end portions comprising the pressing member is attached.
